(19) United States
(12) Patent Application Publication
(10) Pub. No.: US 2004/0122344 A1
(43) Pub. Date: Jun. 24, 2004

(54) Adjustable fit athletic supporter and method therefor

(76) Inventors: Ronald E. Nelson, Cambridge, MN (US); Curt F. Mueller, Prairie du Sac, WI (US)

Correspondence Address: FOLEY & LARDNER, 150 EAST GILMAN STREET, P.O. BOX 1497, MADISON, WI, 53701-1497, US

(21) Appl. No.: 10/327,504

(22) Filed: Dec. 20, 2002

(51) Int. Cl. A61F 13/00

(52) U.S. Cl. 602/61; 602/60

(57) ABSTRACT

An adjustable fit athletic supporter in which the length of the waistband, the length of the leg straps, or both, may be adjusted to closely fit a person. According to another aspect of the invention, the waistband, or leg straps, or both, may bear sizing indicia or numbers to indicate the dimensions of an athletic supporter that closely fits a person. According to yet another aspect of the invention, the dimensions of an athletic supporter that closely fits a person may be used to custom tailor an athletic supporter for that person. An adjustable fit athletic supporter and method therefor according to the invention may more closely fit a person, and may reduce inventory requirements.

### FIELD OF THE INVENTION

The present invention relates generally to the field of articles worn by persons to reduce the likelihood, severity, or exacerbation of injury to the body, and more specifically to athletic supporters.

### BACKGROUND OF THE INVENTION

Participants in strenuous sports and other vigorous activities face the risk of various types of injury. This is especially true in aggressive contact sports or sports involving flying projectiles, such as football, soccer, baseball, or hockey.

One type of injury, which is of particular interest to male athletes, is the risk of impact injury to the genitals. Testicular injuries are painful, often require surgery, and can result in a loss of the organ. Further, testicular bruising in adolescence can result in permanent sterility and infertility problems. There can be little doubt of a broad consensus among male athletes that injuries to the genitals are to be avoided.

It is well known that the risk of impact injury can be reduced by wearing an athletic supporter, and that the risk of impact injury can be reduced even further by wearing such an athletic supporter with a hard athletic cup. For these reasons, the purchase of an athletic supporter is a familiar ritual for male participants in organized sports.

Athletic supporters have been used for many years, and a standard design incorporating an elastic belt, two leg straps, and a pouch is common. However, the practical value of this standard design is limited by real-world considerations such as accurate sizing and inventory management.

A typical sporting goods store may stock athletic supporters in several standardized sizes, such as XS, S, M, L, and XL, where each standardized size is intended to fit a range of actual waist sizes. For example, size M might be intended to fit waist sizes ranging from 32 to 38. However, this standardized size M will actually be a little small for a person with a 38 inch waist and a little large for a person with a 32 inch waist. Thus, the use of standardized sizes means the majority of persons will not be able to buy an athletic supporter that fits well.

It would be possible to improve the fit of athletic supporters by selling them by waist size, without using standardized sizes. For example, a selection of athletic supporters in every waist size from 22 up to 48 inches could be provided. However, this approach would require, for example, 27 different products, much greater than the number of different products required using standardized sizes. The increased number of different products would increase the cost to maintain inventory and to provide shelf space for these products, compared to standardized sizing.

Even standardized sizing does not avoid inventory management problems. The demand for athletic supporters can vary, for example, because of seasonal sports, such as football, or the start of the school year. The relative demand for specific standardized sizes can also fluctuate, as a result of variations in the population of consumers. For these reasons, a retailer may be forced to carry more inventory than would otherwise be desirable in order to maintain sufficient inventory to meet the fluctuations in demand for a particular standardized size.

Another problem which is found in prior athletic supporters is proper sizing of the leg straps. That is, the population of consumers of athletic supporters will not only vary in waist size, they will also vary in leg size. It might be possible to provide athletic supporters to accommodate both variations in waist size and in leg strap size, similar to the way that trousers are sold according to both waist and inseam length. However, this approach would exacerbate inventory management problems, whether sold by waist size or using standardized waist sizing.

Another problem which is found in prior athletic supporters is related to the sizing and inventory management problems. In order to accommodate a wide range of waist and leg sizes using a few standardized sizes, the materials forming the waistband and leg straps are typically chosen to be quite elastic and able to stretch a great deal. However, the more elastic a material, the less support it may be able to provide.

### SUMMARY OF THE INVENTION

The present invention features an athletic supporter having a waistband whose length can be adjusted to closely fit a wide range of waist sizes. Other aspects of the invention feature an athletic supporter with adjustable leg strap sizes, or of a few standardized leg strap sizes, where the athletic supporter may also have a waistband whose length can be adjusted to closely fit a wide range of waist sizes. Yet another aspect of the invention features a custom tailored athletic supporter, and methods of making and selling custom tailored athletic supporters.

In a preferred embodiment, an athletic supporter according to the present invention includes an adjustable size waistband which is long enough to fit a large waist, having two free ends with one free end bearing hook type material and the other free end bearing loop type material. In such a preferred embodiment, excess length from a free end of the waistband can be trimmed to closely fit the waist size of the wearer, and then the waistband may be closed by pressing the hook and loop material on the free ends together.

An embodiment according to another aspect of the invention features an athletic supporter having adjustable size leg straps which are long enough to fit large legs, having two free ends with the first free end bearing hook type material and the second free end bearing loop type material. In such an embodiment, excess length from a free end of each leg strap can be trimmed to closely fit the leg size of the wearer, and then the leg straps may be closed by pressing the hook and loop material on the free ends together.

According to another aspect of the invention, a selection of athletic supporters in a few standardized leg strap sizes is provided, where the athletic supporters include an adjustable size waistband. In this aspect of the invention, a consumer chooses an athletic supporter to closely fit their leg size, then the waistband is adjusted to closely fit their waist size.

Yet another aspect of the invention features methods of making an athletic supporter having dimensions which are tailored to a specific person. A method of making an athletic supporter according to the present invention includes providing measurements of a specific person, cutting material to fit those measurements, and sewing the material together to form a custom tailored athletic supporter. Another method of making an athletic supporter according to the present invention includes providing an athletic supporter having an adjustable size waistband and adjustable size leg straps, where the waistband and leg straps bear markings to indicate their length, obtaining the measurements of a specific person using the markings on the waistband and leg straps, cutting material to fit those measurements, and sewing the material together to form a custom tailored athletic supporter.

Further objects, features, and advantages of the invention will be apparent from the following detailed description when taken in conjunction with the accompanying drawings.

### BRIEF DESCRIPTION OF THE DRAWINGS

In the drawings:

FIG. 1 is a front perspective view of an athletic supporter according to the invention.

FIG. 2 is a front view of the athletic supporter according to the invention laid out flat.

FIG. 3 is a rear view of the athletic supporter according to the invention laid out flat.

FIG. 4 is a side perspective view of the athletic supporter according to the invention.

FIG. 5 is a side perspective view of the athletic supporter according to the invention after trimming excess length.

FIG. 6 is a front view of the athletic supporter according to the invention.

FIG. 7 is a side view of the athletic supporter according to the invention.

FIG. 8 is a front view of a first alternate embodiment of an athletic supporter according to the invention laid out flat.

FIG. 9 is a rear view of the athletic supporter of FIG. 8 laid out flat.

FIG. 10 is a front view of a second alternate embodiment of an athletic supporter according to the invention laid out flat.

### DETAILED DESCRIPTION OF THE INVENTION

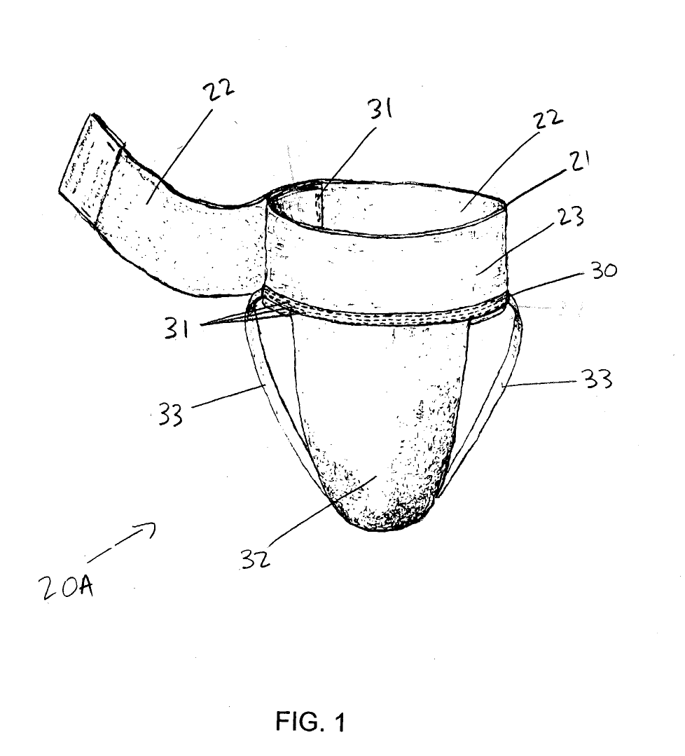

With reference to the drawings, a preferred embodiment of an athletic supporter according to the present invention is indicated generally at 20A in FIG. 1. Such an athletic supporter includes a waistband 21 having an inner surface 22 and an outer surface 23. The waistband 21 is preferably comprised of hook compatible (loop type) elastic. In a preferred embodiment the inner surface 22 of the waistband 21 carries loops of the type which will adhere to hook type material when pressed together.

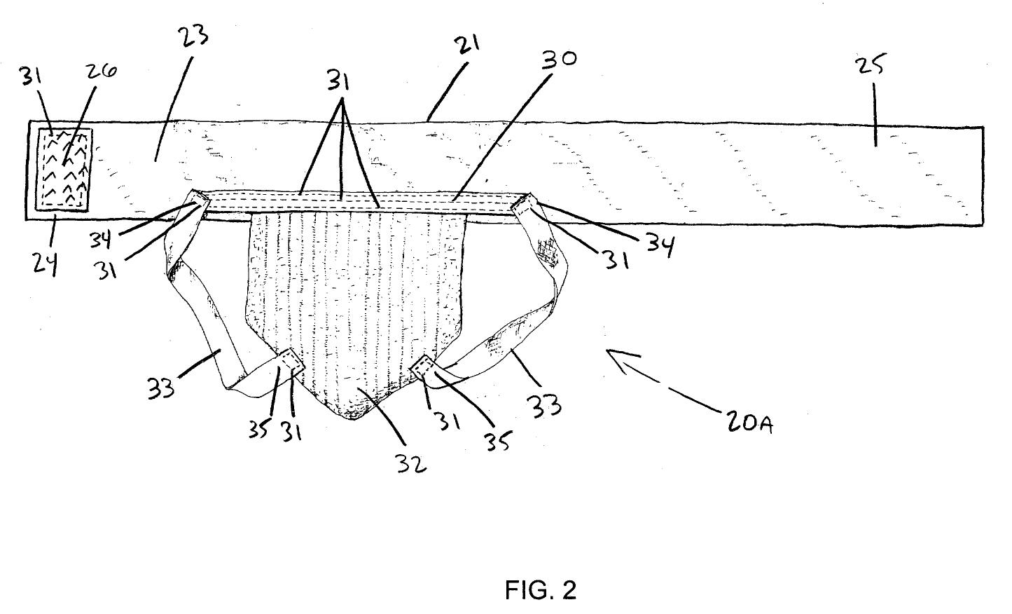

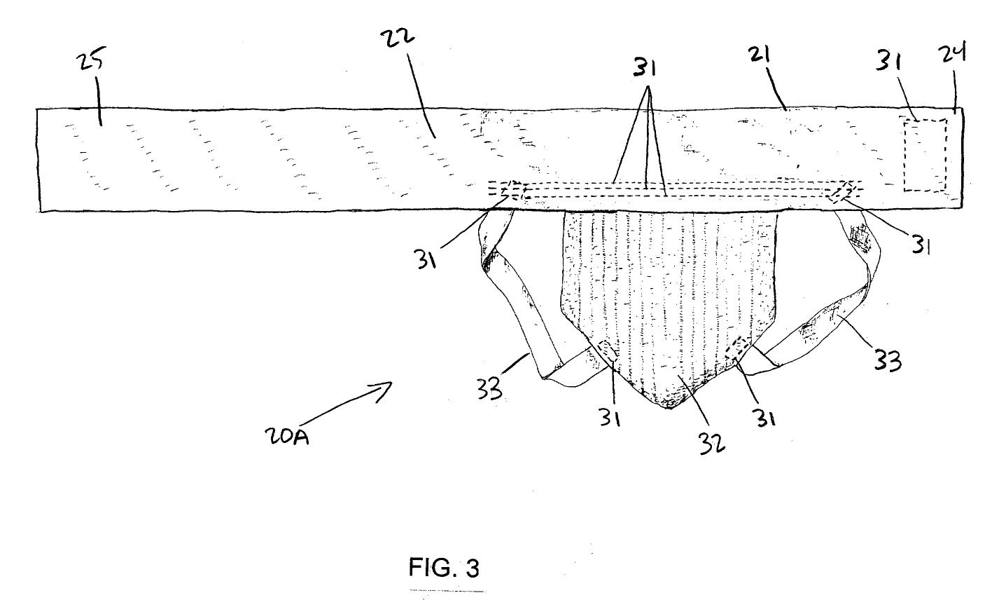

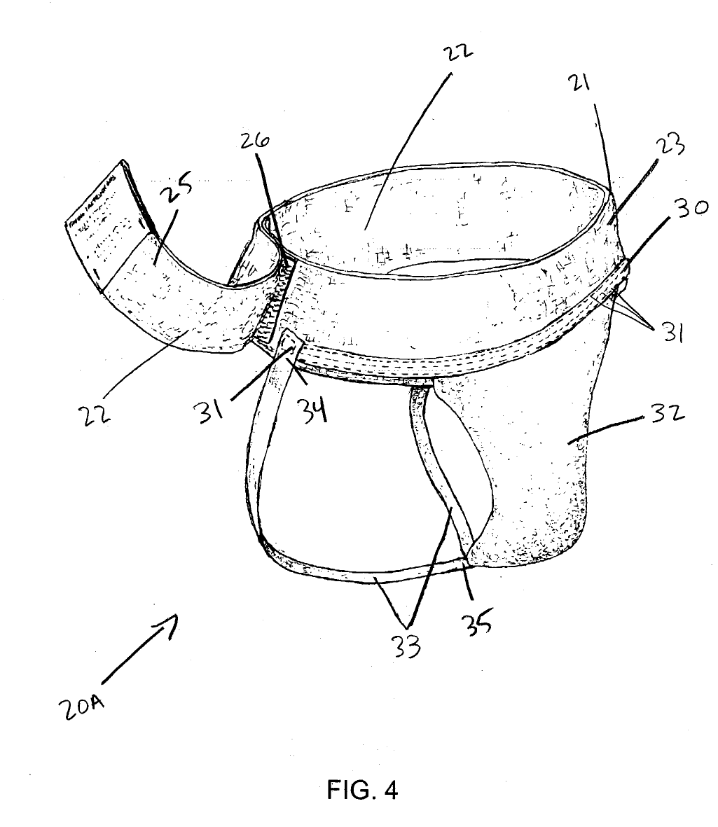

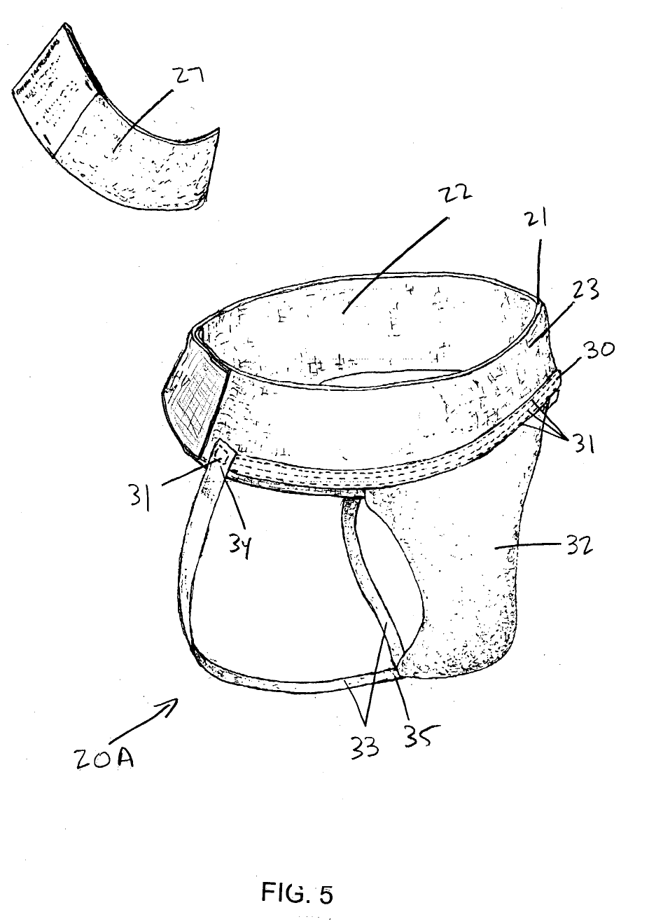

As best shown in FIGS. 2 and 3, the waistband 21 preferably has a fixed length free end 24, and an adjustable length free end 25. In a preferred embodiment, the fixed length free end 24 bears an attachment patch 26 made of hook type material. As best shown in FIG. 4, the inner surface 22 of the adjustable length free end 25 bears loop type material, so that it may be pressed against the attachment patch 26 to fasten the waistband 21 about the waist of the wearer. As best shown in FIG. 5, the length of the adjustable length free end 25 may be adjusted by cutting off the excess waistband 27, for example using a scissors or knife.

An athletic supporter according to the invention may be otherwise of conventional construction. A reinforcement strip 30 may be fastened to the waistband 21, for example using stitches 31, although such a reinforcement strip 30 is not required. The athletic supporter includes a pouch 32 which is fastened, for example using stitches 31, to the waistband 21 and the reinforcement strip 30, if present.

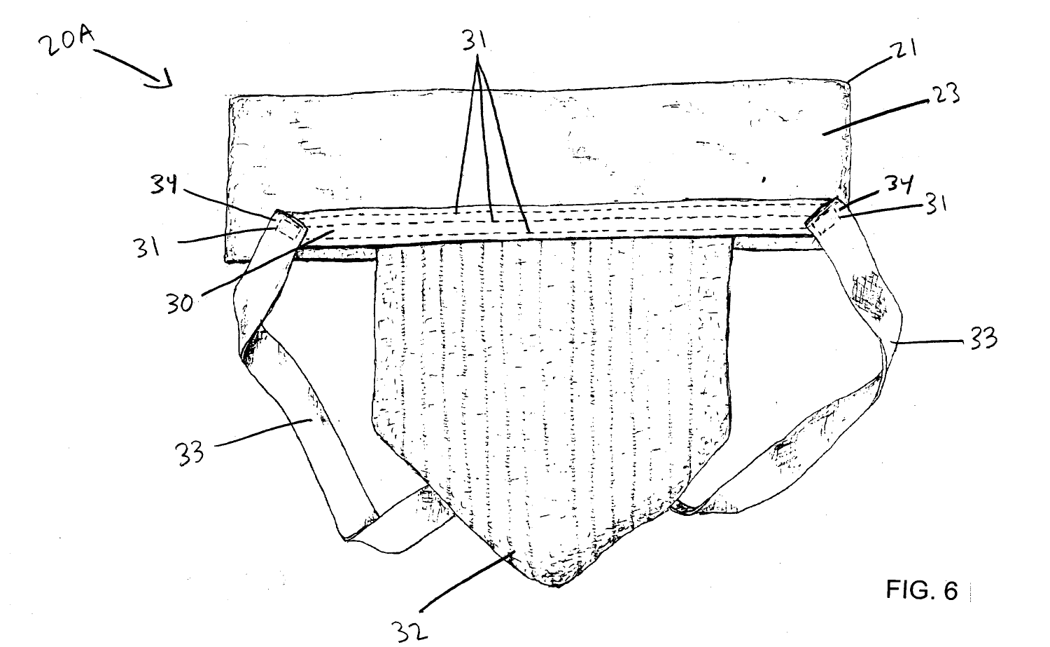

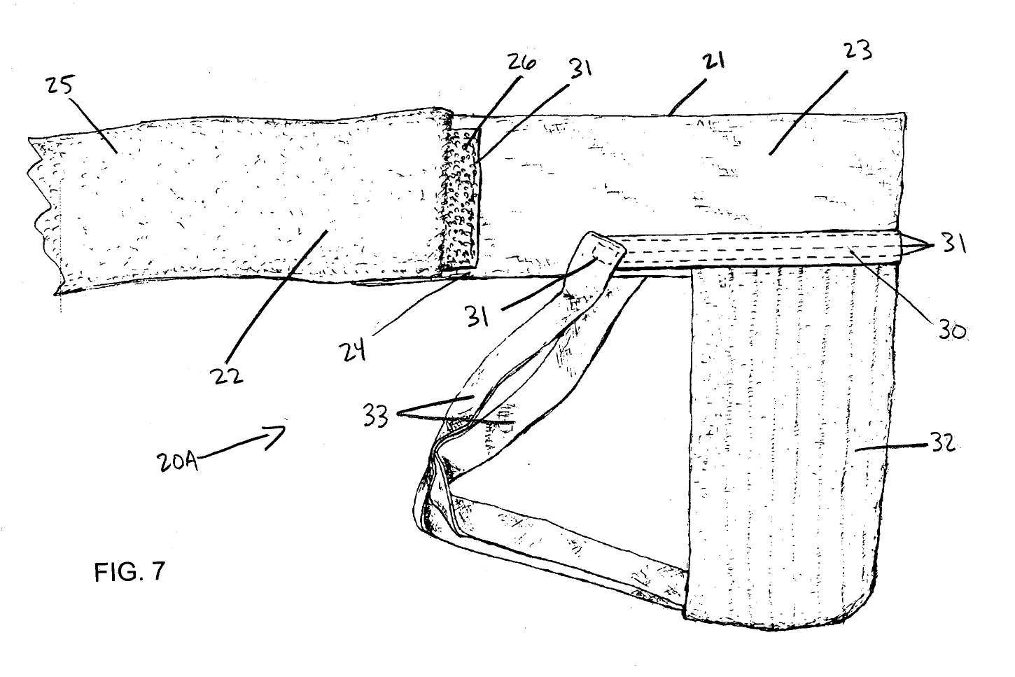

The preferred embodiment of an athletic supporter 20A includes two leg straps 33. The upper end 34 of each leg strap 33 is fastened, for example using stitches 31, to the waistband 21, and may also be fastened to the reinforcement strip 30, if present. The lower end 35 of each leg strap 33 is fastened, for example using stitches 31, to the pouch 32. FIGS. 6 and 7 are additional illustrations of the construction of such an athletic supporter.

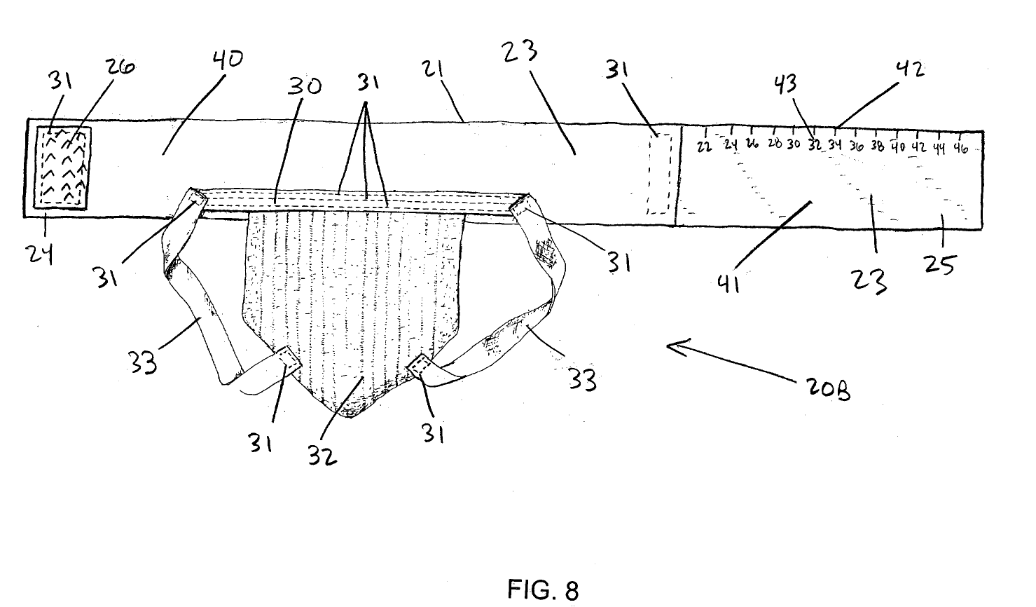

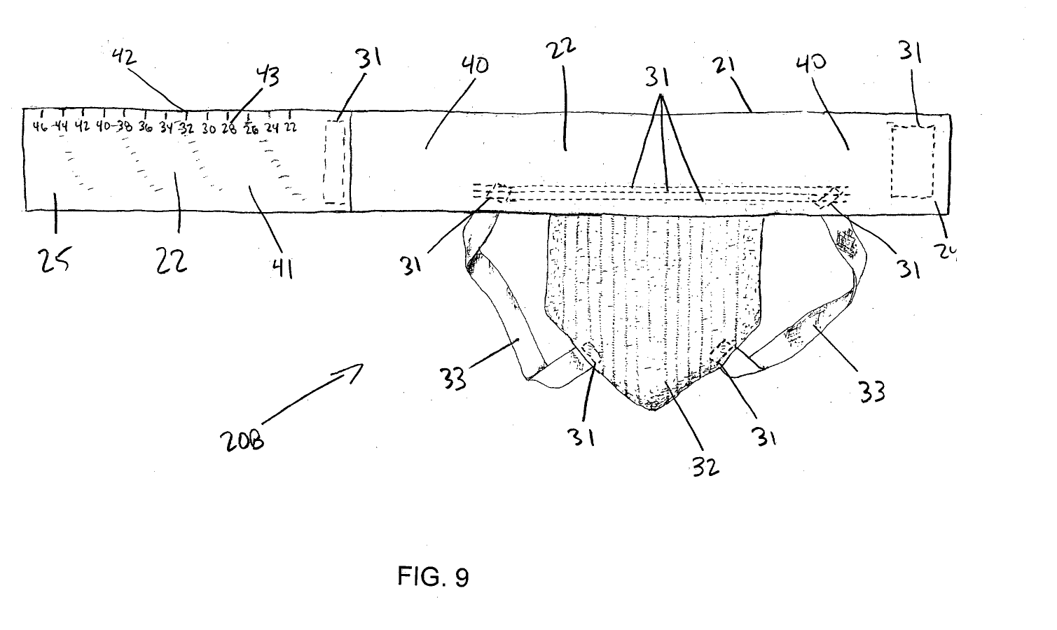

FIGS. 8 and 9 show another embodiment 20B of an athletic supporter according to the present invention. In such an athletic supporter, the waistband may include a first waistband section 40 and a second waistband section 41. The first waistband section 40 may include a fixed length free end 24, and is preferably comprised of standard elastic material, although this is not necessary and other elastic, directionally elastic, or inelastic materials may be used. The second waistband section 41 may include an adjustable length free end 25, and is preferably comprised of hook compatible (loop type) elastic, although this is not necessary and other elastic, directionally elastic, or inelastic materials may be used. The inner surface 22 of the second waistband section 41 preferably carries loops of the type which will adhere to hook type material when pressed together, although this is not required and other means may be used to join the fixed length free end 24 to the adjustable length free end 25, such as stitching, glue, zippers, straps, rivets, buckles, buttons, thermal bonding, or other means known in the art.

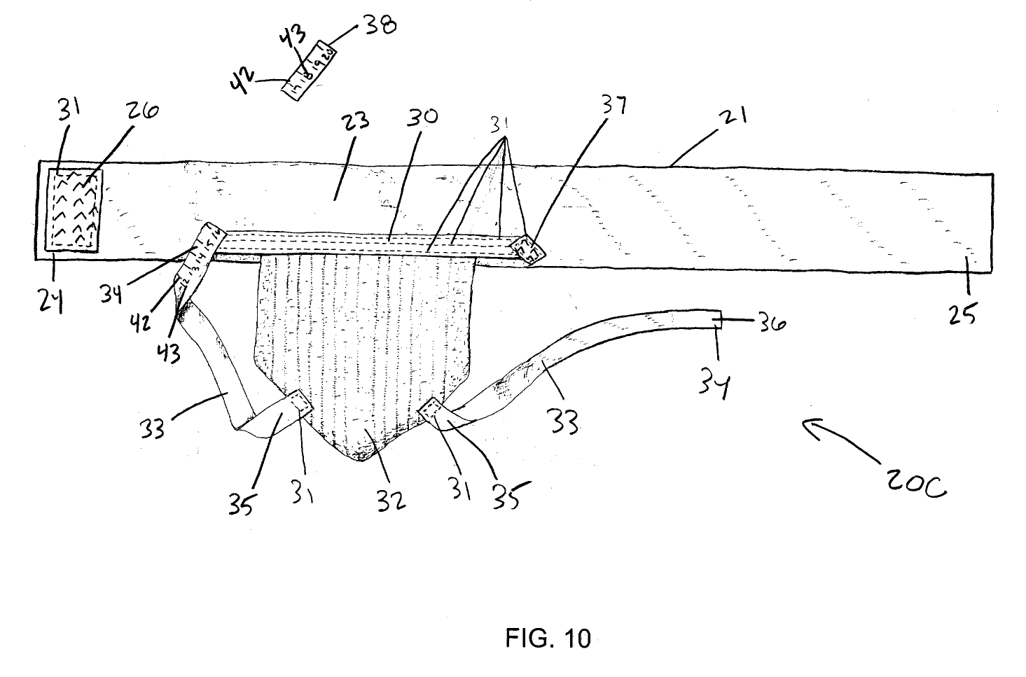

FIG. 10 shows a further embodiment 20C of an athletic supporter according to the present invention. In such an athletic supporter, the upper end 34 of each leg strap 33 is a free end 36 and the lower end 35 of each leg strap 33 is fastened to the pouch 32, for example using stitches 31. In such an athletic supporter, at least the free end 36 of each leg strap 30 bears loop type material. In such an athletic supporter, the leg straps 30 may be passed around the legs of the wearer and detachably attached to the waistband by pressing the free end 36 of each leg strap 30 against an attachment patch 37 made of hook material which is fastened, for example using stitches 31, to the waistband 21 and the reinforcement strip 30, if present. As shown in FIG. 10, the length of the free end 36 of each leg strap may be adjusted by cutting off the excess leg strap 38, for example using a scissors or knife.

As best shown in FIGS. 8-10, the waistband 21, or the leg straps 30, or both, may bear size markings, for example size indicia 42 or size numbers 43 or both, although this is not required. The waistband size markings may be read to obtain the ideal waistband length for a particular wearer after adjusting the length of the waistband by cutting off the excess waistband 27. Similarly, the leg strap size markings may be read to obtain the ideal leg strap lengths for a particular wearer after adjusting the length of each leg strap 33 by cutting off the excess leg strap 38. According to another aspect of the invention, a particular wearer may obtain these ideal waistband length measurements or leg strap measurements or both measurements, for use in manufacturing a custom-fitted athletic supporter for that particular wearer. Because a custom-fitted athletic supporter will fit its wearer very closely, it may be comprised of material which is less elastic, and which therefore provides better support, compared to the elastic material used in an athletic supporter which is made to fit a range of sizes.

There are various possibilities with regard to alternative embodiments and methods including an athletic supporter according to the invention.

For example, an athletic supporter according to the invention may allow adjustment of the size of both the waistband and the leg straps.

Although in a preferred embodiment, hook and loop type material may be used to detachably attach the ends of the waistband together, and to detachably attach the upper end of the leg straps to the waistband, other means may be used. Although in a preferred embodiment, hook and loop material is used to form detachable attachments, permanent attachments may be formed. For example, stitching, glue, zippers, straps, rivets, buckles, buttons, thermal bonding, or other means may be used instead of, or in addition to, hook and loop type material.

Although in a preferred embodiment, hook compatible elastic material may be used for the waistband or the leg straps, other materials could be used. For example, relatively inelastic or directionally elastic fabric bearing hook compatible material could be used instead of, in addition to, or in combination with, hook compatible elastic material.

Although in a preferred embodiment, the attachment patch is made of hook type material attached to the outside surface of the waistband, with hook compatible (loop) material on the inside surface of the waistband, a variety of other arrangements are possible. For example, the attachment patch could be loop type material, with hook type material on the inside surface of the waistband. The attachment patch, either hook or loop type material, could be attached to the inside surface of the waistband, with complementary hook or loop type material on the outside surface of the waistband.

Although in a preferred embodiment, stitches are used to fasten together various pieces of an athletic brace according to the invention, (such as fastening the attachment patch to the reinforcement strip or waistband, fastening the reinforcement strip to the waistband, or fastening the leg straps to the pouch and reinforcement strip), other means could be used. For example, stitching, glue, zippers, straps, rivets, buckles, buttons, thermal bonding, or other means known in the art could be used alone or in combination.

It is understood that the invention is not confined to the embodiments set forth herein as illustrative, but embraces all such forms thereof as come within the scope of the following claims.

What is claimed is:

1. An athletic supporter comprising:
(a) a waistband, having a first free end bearing an attachment patch made of hook type material, and a second free end made of hook compatible elastic;
(b) a pouch fastened to the waistband;
(c) a first leg strap having a lower end fastened to the pouch and an upper end fastened to the waistband; and
(d) a second leg strap having a lower end fastened to the pouch and an upper end fastened to the waistband.

2. The athletic supporter of claim 1 further comprising a reinforcement strip fastened to the waistband.

3. The athletic supporter of claim 1 wherein at least one free end of the waistband bears sizing numbers.

4. The athletic supporter of claim 1 wherein at least one free end of the waistband bears sizing indicia.

5. An athletic supporter comprising:
(a) a waistband bearing a first leg strap attachment patch formed of hook type material, and a second leg strap attachment patch formed of hook type material
(b) a pouch fastened to the waistband;
(c) a first leg strap having a lower end fastened to the pouch and an upper end which includes loop type material; and
(d) a second leg strap having a lower end fastened to the pouch and an upper end which includes loop type material.

6. The athletic supporter of claim 5 further comprising a reinforcement strip fastened to the waistband.

7. The athletic supporter of claim 5 wherein at least one free end of at least one of the first leg strap and the second leg strap bears sizing numbers.

8. The athletic supporter of claim 5 wherein at least one free end of at least one of the first leg strap and the second leg strap bears sizing indicia.

9. A method of marketing athletic supporters, comprising:
(a) providing a first athletic supporter including an adjustable size waistband, a pouch fastened to the waistband, a first leg strap of a first specified length having a first end fastened to the waistband and a second end fastened to the pouch, and a second leg strap of a first specified length having a first end fastened to the waistband and a second end fastened to the pouch;
(b) providing a second athletic supporter including an adjustable size waistband, a pouch fastened to the waistband, a first leg strap of a second specified length having a first end fastened to the waistband and a second end fastened to the pouch, and a second leg strap of a second specified length having a first end fastened to the waistband and a second end fastened to the pouch; and
(c) offering the first and second athletic supporters for sale in close proximity to one another.

10. The method of claim 9 further comprising providing a third athletic supporter including an adjustable size waistband, a pouch fastened to the waistband, a first leg strap of a third specified length having a first end fastened to the waistband and a second end fastened to the pouch, and a second leg strap of a third specified length having a first end fastened to the waistband and a second end fastened to the poucha reinforcement strip fastened to the waistband.

11. The method of claim 9 wherein at least one of the first and second athletic supporters bears sizing numbers on its waistband.

12. The method of claim 9 wherein at least one of the first and second athletic supporters bears sizing indicia on its waistband.

13. A method of adjusting the size of an athletic supporter to fit a particular wearer of a specified waist size, comprising:
(a) providing an athletic supporter which includes a waistband of a length greater than the specified waist size of the particular wearer and having a first free end bearing hook type material and a second free end bearing loop type material;
(b) placing the athletic supporter on the body of the particular wearer as the athletic supporter would normally be worn, such that the waistband wraps about the waist of the particular wearer with the second free end overlapping the first free end; and
(c) cutting off any excess waistband from at least one of the first or second free ends.

14. The method of claim 13 wherein the waistband of the athletic supporter bears size indicia and further comprising the step of determining the ideal waist size of the particular wearer using the size indicia.

15. The method of claim 14 further comprising the step of using the ideal waist size of the particular wearer to manufacture a custom-fitted athletic supporter.

16. The method of claim 13 wherein the waistband of the athletic supporter bears size numbers and further comprising the step of determining the ideal waist size of the particular wearer using the size numbers.

17. The method of claim 16 further comprising the step of using the ideal waist size of the particular wearer to manufacture a custom-fitted athletic supporter.

18. A method of adjusting the size of an athletic supporter to fit a particular wearer of a specified leg size, comprising:
(a) providing an athletic supporter which includes a waistband bearing attachment patches made of hook type material, and leg straps of a length greater than the specified waist size of the particular wearer and having a free end bearing loop type material;
(b) placing the athletic supporter on the body of the particular wearer as the athletic supporter would normally be worn, such that at least one leg strap wraps about a leg of the particular wearer with the free end of the leg strap overlapping an attachment patch on the waistband; and
(c) cutting off any excess leg strap from that leg strap.

19. The method of claim 18 wherein the leg strap bears size indicia and further comprising the step of determining the ideal leg strap size of the particular wearer using the size indicia.

20. The method of claim 19 further comprising the step of using the leg strap size of the particular wearer to manufacture a custom- fitted athletic supporter.

21. The method of claim 18 wherein the leg strap bears size numbers and further comprising the step of determining the ideal leg strap size of the particular wearer using the size numbers.

22. The method of claim 21 further comprising the step of using the ideal leg strap size of the particular wearer to manufacture a custom-fitted athletic supporter.
